(19) United States
(12) Patent Application Publication
(10) Pub. No.: US 2015/0140111 A1
(43) Pub. Date: May 21, 2015

(54) PREPARATIONS OF EFFERVESCENT FORMULATIONS COMPRISING SECOND AND THIRD GENERATION CEPHALOSPORIN AND USES THEREOF

(71) Applicant: Mahmut BILGIC, Istanbul (TR)

(72) Inventor: Mahmut BILGIC, Istanbul (TR)

(21) Appl. No.: 14/309,273

(22) Filed: Jun. 19, 2014

Related U.S. Application Data
(63) Continuation of application No. 13/668,922, filed on Nov. 5, 2012, now abandoned.
Application No. 13/668,922 is a continuation-in-part of application No. PCT/TR2011/000124, filed on May 13, 2011.
Application No. PCT/TR2011/000124 is a continuation-in-part of application No. PCT/TR2011/000127, filed on May 2, 2011.
Application No. PCT/TR2011/000127 is a continuation-in-part of application No. PCT/TR2011/000129, filed on May 2, 2011.
Application No. PCT/TR2011/000129 is a continuation-in-part of application No. PCT/TR2011/000131, filed on May 2, 2011.

(30) Foreign Application Priority Data
May 4, 2010 (TR) 2010/03543
May 4, 2010 (TR) 2010/03546
May 4, 2010 (TR) 2010/03547
May 14, 2010 (TR) 2010/03854

(51) Int. Cl. A61K 9/46; A61K 47/26; A61K 47/02; A61K 31/546

(52) U.S. Cl. CPC A61K 9/0007; A61K 31/546; A61K 47/26; A61K 47/02
USPC 424/501; 424/43; 514/202; 514/207

(57) ABSTRACT

The invention relates to effervescent pharmaceutical dosage forms including cefdinir as the active agent, and their preparation. The invention also relates to effervescent formulations including ceftibuten and/or its pharmaceutically acceptable salts, hydrates, solvates, esters, amorphous and crystal forms and/or a combination thereof. The invention also relates to pharmaceutical compositions including (Z)-3-Carboxymethyl-7-(2-(2-furyl)-2-methoxyiminoacetylamino)-3-sefem-4-carboxylic acid which is named cefuroxime axetil or any pharmaceutically acceptable derivative thereof, and the use of these compositions in the treatment of bacterial infections. Lastly, the invention relates to pharmaceutical formulations including a third generation cephalosporin together with clavulanic acid and/or derivatives thereof as the active agents.

### CROSS-REFERENCE TO RELATED APPLICATIONS

This application is a continuation of U.S. application Ser. No. 13/668,922, filed Nov. 5, 2012, which is a continuation-in-part of PCT/TR2011/000127, PCT/TR2011/000129, PCT/TR2011/000131, filed May 2, 2011, and PCT/TR2011/000124, filed May 13, 2011, which are incorporated herein by reference in their entireties. U.S. application Ser. No. 13/668,922 is entitled to and claims priority benefits to application Serial Numbers TR2010/03543, TR2010/03546, TR2010/03547, filed May 4, 2010, and TR2010/03854, filed May 14, 2010.

### BACKGROUND OF THE INVENTION

### Cefdinir

The chemical structure of the molecule cefdinir, which has the chemical name (6R,7R)-7-[[(2Z)-(2-amino-4-thiazolil)(hydroxyimino)acetyl]amino]-3-ethenyl-8-oxo-5-thia-1-azabicyclo[4.2.0]oct-2-en-2-carboxylic acid and was first disclosed in the patent numbered BE897864, is shown in Formula I. This molecule, which is a third generation cephalosporin, is indicated for the treatment of many diseases caused by gram positive and gram negative bacteria.

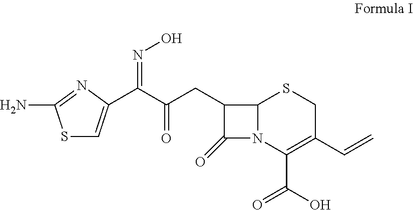 [SMILES: C=CC1=C(C(=O)O)N2C(=O)C(CC(=O)/C(=N\O)c3csc(N)n3)C2SC1]

Cefdinir which physically appears as a white powder has very poor solubility in common organic solvents such as methanol, ethanol, and acetonitrile and in water. Cefdinir do not get wet or dissolve in water owing to its hydrophobic character. Moreover, cefdinir has a very bitter taste that it is difficult to mask it with flavoring agents or taste regulating agents.

The product under the name OMNICEF® is available in capsule and suspension forms on the market. Administration of solid oral dosage forms such as capsules, pills and tablets by oral route generally causes difficulty in swallowing for geriatric and pediatric patients. Although alternatively developed suspension dosage forms indicate higher bioavailability, the use of this dosage form brings along the possibility of excessive and/or uncontrolled dose intake particularly in pediatric and geriatric patients.

In the solid oral and suspension dosage forms, some methods have been tried in order to eliminate the bitter taste of cefdinir. In the tablets containing coating, it has been tried to mask the bitter taste of cefdinir with the help of the coating material. However, cefdinir has such a bitter taste that coating materials are unable to mask it. On the other hand, the coatings in the suspension dosage forms provide a solution for low solubility problem of cefdinir while they remain incapable of masking the bitter taste of it.

Alternatively, water dispersible forms are preferred since they are more convenient to use for geriatric and pediatric patients and they solve the solubility problem of cefdinir. Although some excipients such as taste regulating agent, aroma and/or flavoring agents are used to eliminate the bitter taste of cefdinir, a desirable and pleasant taste of it cannot be achieved.

Accordingly, it is clearly seen that the problems such as difficulty in swallowing or low solubility of cefdinir can be eliminated to some extent. However, masking the bitter taste of cefdinir is a challenging task to overcome.

As it can be seen, new dosage forms and formulations need to be developed so as to provide reliable and user friendly dosage form alternatives for pediatric and geriatric patients, to overcome the solubility problems of cefdinir and especially, to eliminate the bitter taste of cefdinir.

### Ceftibuten

Ceftibuten, which has the chemical name (+)-(6R,7R)-7-([(Z)-2-(2-amino-4-thiazolyl)-4-carboxycrotonamido]-8-oxo-5-thia-1-azabicyclo[4.2.0]oct-2-ene-2-carboxylic acid, was first disclosed in the patent numbered U.S. Pat. No. 4,634,697. It was indicated in the patents numbered U.S. Pat. No. 4,933,443 and U.S. Pat. No. 4,812,561 that ceftibuten is stable in dihydrate and trihydrate forms. Moreover, it was indicated in said patents that dihydrate and trihydrate forms of ceftibuten can be filled into hard gelatin capsules for administration by the oral route.

However, the use of such oral dosage forms is not preferred since they become quite large in size and this made the use of this dosage form inconvenient in patients with dysphagia, particularly in pediatric and geriatric patients.

The suspension forms have been alternatively developed to overcome said problems and dry powder formulations comprising ceftibuten which are suitable for use in suspension form were disclosed in the patent numbered EP0642344. However, they are not preferred since the potential of high and/or uncontrolled dose intake and they are not physically and chemically stable. Alternatively, the use of reliable and user-friendly effervescent forms is suggested.

The physical properties of the final product in the effervescent form such as hardness and dissolution time can change according to the formulations developed. These changes in the physical properties of the effervescent formulations are of great importance on the preparation and usage of the final product. In cases where the effervescent formulations comprising ceftibuten are compressed into effervescent tablet, dissolution time has an important effect on the disintegration of these effervescent tablets in water.

In cases where the effervescent formulations comprising ceftibuten are compressed into effervescent tablet, increase in the tablet hardness leads to the increase in dissolution time of the ceftibuten formulations in water. This causes increase of time period it takes to get the drug ready for use and thus leads to an inefficient delivery of the drug.

As is seen, new formulations comprising ceftibuten are required to be developed in order to provide new formulations which are quickly dispersed in water during use and convenient to administer effectively.

The inventors have surprisingly found that said problems with formulations of cefdinir and ceftibuten in the prior art can be solved by the effervescent formulations prepared according to the subject of the present invention.

### Cefuroxime Axetil

Cefuroxime axetil shown in Formula II is a second generation cephalosporin which was first disclosed in the patent numbered U.S. Pat. No. 3,974,153.

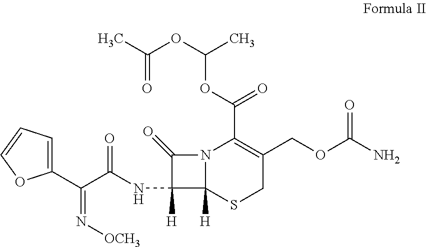

Cefuroxime axetil is a broad spectrum cephalosporin antibiotic against gram negative and gram positive bacteria. Three different polymorphs of it have been described in the prior art. These are the crystal form having a melting point of approximately 180° C., the amorphous form having a melting point of approximately 135° C. and the amorphous form having a melting point of approximately 70° C. Cefuroxime axetil in both crystalline form and the amorphous form has a tendency to form a gelatinous mass when contacted with water. This gel formation leads to poor dissolution of cefuxotime axetil and thus low absorption of it.

Cefuroxime axetil has a very bitter taste which is highly durable and which cannot be easily masked by adding sweeteners, taste regulating agents and flavors. In the prior art, coatings have been suggested for both eliminating the extremely bitter taste of cefuroxime axetil and for preventing contact of cefuroxime axetil with water and form a gelatinous mass.

Antibiotics such as cefuroxime axetil for oral administration are generally in the form of granules which can be administered as a suspension since the suspension dosage forms for administration by the oral route are advantageous in respect of their higher dissolution and lower bitterness. Moreover, they do not present the problems encountered in solid dosage forms. For instance, the patent numbered US20030161888, discloses a composition comprising cefuroxime axetil in particulate form formulated as an oral suspension, the particles being provided with integral coatings of lipid or mixture of lipids which are insoluble in water and which disperse or dissolve on contact with gastrointestinal fluid characterized in that said composition comprises a sweetener system and a texture modifier. In this patent application, it is aimed to develop a cefuroxime axetil formulation in suspension form which is easy to swallow and provides to mask the significant bitter taste of cefuroxime axetil and to improve the mouth feel.

However, the coated particles are prepared by atomizing a dispersion of particulate cefuroxime axetil in the molten lipid and cooling the coated particles. This process is a complicated and time consuming process. Moreover, lipid coating may retard the dissolution of cefuroxime axetil and delay its absorption. Furthermore, they are not sufficient to prevent the gelling problem entirely and to mask the bitter taste of cefuroxime axetil.

As is seen, it is required to develop new formulations comprising cefuroxime axetil in suspension form which are prepared by using an easy conventional method, which can overcome the dissolution problem by preventing the gelling while providing a high absorption of it and which eliminates the bitter taste of cefuroxime axetil.

The inventors have surprisingly found that the formulations pertaining to present invention comprising cefuroxime axetil in suspension form, which are produced easily, prevent not only the gelling problem but also the bitter taste of cefuroxime axetil.

### Third Generation Cephalosporin and Clavulanic Acid

Third generation cephalosporins which are broad spectrum antibiotics are known to have antibacterial effect both on gram positive and gram negative bacteria. The compounds known as cefdaloxim, cefdinir, cefditoren, cefetamet, cefixime, cefmenoxime, cefodizime, cefotaxime, cefovecin, cefpimizole, cefpodoxime, cefteram, ceftibuten, ceftiofur, ceftiolene, ceftizoxime, ceftriaxone, cefetamet belong to the group of the third generation cephalosporins.

Clavulanic acid is a beta-lactamase inhibitor. Clavulanic acid and derivatives thereof (e.g. its salts such as potassium clavulanate) are known as beta-lactamase inhibitors which withstand the beta-lactamase-originated resistance mechanism by suppressing the activity of beta-lactamase enzymes. The patent numbered EP0593573 discloses a formulation relating to suspension forms comprising beta-lactam antibiotics, especially amoxicillin and ampicillin, with beta lactamase inhibitors.

However, suspension forms are not preferable since they have the potential of high and/or uncontrolled dose intake; they have problems in their physical and chemical stability, they have high manufacture costs and they cause problems in use and carrying. Alternatively, water dispersible tablets have been developed in order to improve the bioavailability of the antibiotic and to have them disintegrate faster. The patent application numbered in EP 0282200 discloses dispersible formulations comprising an amphoteric beta-lactam antibiotic and as disintegrants, a cellulose product and low-substituted hydroxypropylcellulose.

However, some problems have been observed in water dispersible formulations comprising third generation cephalosporin antibiotics due to low solubility of them. Although the third generation cephalosporins have broad spectrum, their water solubility is low, which leads to low dissolution and absorption of the third generation cephalosporin antibiotic and slow dispersion of their water dispersible formulations.

Furthermore, clavulanic acid and its pharmaceutically acceptable derivatives are highly sensitive to conditions such as humidity and pH, for this reason it is very difficult to prepare stable and water soluble formulations wherein clavulanic acid or its pharmaceutically acceptable derivatives and third generation cephalosporins are used in combined form.

As is seen, new formulations comprising third generation cephalosporin antibiotics and clavulanic acid derivatives are required to be developed in order to provide new dosage forms which disperse quickly by providing the entire dissolution of the antibiotic in water.

The inventors have surprisingly found that the water soluble dosage forms comprising third generation cephalosporin antibiotics and clavulanic acid derivatives pertaining to the present invention overcome the problems encountered in the prior art.

### DESCRIPTION OF THE INVENTION

### Formulations Comprising Cefdinir

The subject of the present invention is effervescent formulations comprising cefdinir and the procedures related to their preparation. Surprisingly, it has been found that the effervescent formulations comprising cefdinir, wherein two different taste regulating agents are used and the ratio of the first taste regulating agent having 31-90% (w/w) water solubility at 25° C. to the second taste regulating agent having 5-30% (w/w) water solubility at 25° C. is in the range of 5:1 to 1:1, have achieved to increase the solubility of cefdinir and eliminate the bitter taste of cefdinir.

The inventors have found that these effervescent formulations developed pertaining to the present invention can be administered by pediatric and geriatric patients easily, can lead to the increase in the solubility of cefdinir and can mask the bitter and durable taste of cefdinir entirely.

The choice of the taste regulating agent with respect to their water solubility values enables fine tuning of the solubility problem of cefdinir and the ratio of these two agents provides effective taste masking of the formed solution.

Accordingly, the first aspect of the present invention is the effervescent formulations comprising cefdinir as the active agent in which two different taste regulating agents are used and the ratio of the first taste regulating agent having 31-90% (w/w) water solubility at 25° C. to the second taste regulating agent having 5-30% (w/w) water solubility at 25° C. is in the range of 5:1 to 1:1.

The term “effervescent formulations” present in the text comprises effervescent tablets, effervescent granules and effervescent powders.

The term “taste regulating agent having 31-90% (w/w) water solubility at 25° C.” means that the amount of taste regulating agent dissolved in 100 g water is in the range of 31-90 g at 25° C.

The term “taste regulating agent having 5-30% (w/w) water solubility at 25° C.” means that the amount of taste regulating agent dissolved in 100 g water is in the range of 5-30 g at 25° C.

Cefdinir that can be used in the effervescent formulations, which are the subject of the present invention, can be in the form of its solvates, hydrates, enantiomers, racemates, organic salts, inorganic salts, polymorphs, crystal and amorphous forms or in free form and/or combination thereof.

The first taste regulating agent that can be used in effervescent formulations of the present invention can be selected from, but not limited to, a group comprising dextrose, fructose, glucose, lactitol, maltitol, maltose, sorbitol, saccharine sodium, sodium cyclamate, sodium chloride, potassium chloride, sucrose and xylitol or combinations thereof. Preferably, sodium chloride is used as the first taste regulating agent in the formulations according to the present invention.

The second taste regulating agent that can be used in effervescent formulations of the present invention can be selected from, but not limited to, a group comprising acesulfame, aspartame, saccharine and sucralose, or combinations thereof. Preferably, sucralose is used as the second taste regulating agent in the formulations according to the present invention.

According to this, another aspect of the present invention is the effervescent formulations comprising cefdinir as the active agent in which sodium chloride and sucralose are used as taste regulating agents and the ratio of sodium chloride to sucralose is in the range of 5:1 to 1:1.

The inventors have found that the ratio of active agent to the taste regulating agent comprising two different agents is significant in masking the bitter taste of cefdinir. Therefore, it has been observed that unpleasant taste of cefdinir is successfully masked in the formulations in which the ratio of cefdinir to the combination of taste regulating agents is in the range of 10:1 to 1:1, preferably 8:1 to 1:1 and more preferably 5:1 to 2:1.

According to this, another aspect of the present invention is effervescent formulations wherein the ratio of cefdinir to the combination of taste regulating agents is in the range of 10:1 to 1:1, preferably 8:1 to 1:1 and more preferably 5:1 to 2:1.

In the present invention, the inventors have also found that the effervescent formulations comprising cefdinir, in which organic base is used in addition to cefdinir and the ratio of cefdinir to the organic base is in the range of 5:1 to 1:5, provide dissolution of cefdinir in water entirely. Therefore, the developed effervescent formulations pertaining to the present invention have both provided a high solubility of cefdinir in water and eliminated the unpleasant taste of cefdinir to a large extent.

According to this, another aspect of the present invention is the effervescent formulations comprising cefdinir in which organic base is used in addition to cefdinir and the ratio of cefdinir to the organic base is in the range of 5:1 to 1:5.

In the effervescent formulations pertaining to the present invention, primary amines, secondary amines, tertiary amines and/or heterocyclic compounds containing nitrogen can be used as the organic base in the present invention.

The organic base that is to be used in the formulation can be selected from, but not limited to, a group comprising ethanolamine, isopropanolamine, 1-dioxy-1-methylamino-sorbitol, 1-dioxy-1-methylamino-D-glucitol, tris(hydroxymethyl)aminomethane, N-(Tri(hydroxymethyl)methyl)glycine, N,N-Bis(2-hydroxyethyl)glycine, 2-methyl aminophenol. Preferably, 1-dioxy-1-methylamino-sorbitol and tris(hydroxymethyl)aminomethane are used.

Another aspect of the present invention is the effervescent formulations comprising pharmaceutically acceptable excipients in addition to the active agent cefdinir.

Various excipients selected from, but not limited to, a group comprising binders, lubricants, humectants, disintegrants, diluents, effervescent acid and effervescent base can be used in the effervescent formulation pertaining to the present invention apart from cefdinir, taste regulating agent and the organic base.

The binder that can be used in the effervescent formulations pertaining to the present invention can be selected from, but not limited to, a group comprising ethyl cellulose, gelatine, hydroxyethyl cellulose, hydroxymethyl cellulose, hydroxypropyl cellulose, hypromellose, magnesium aluminum silicate, methyl cellulose, povidone. Preferably, povidon is used in scope of the present invention.

The inventors have found that the dissolution time of the pharmaceutical formulation when 1-8% binder is used is shorter compared to the formulations comprising a higher amount of binder.

According to this, another aspect of the present invention is effervescent formulations which comprise the binder in the ratio of 1-8%, preferably in the ratio of 1-7%, most preferably in the ratio of 2-5% with respect to the total weight of the composition and cefdinir as the active agent.

The inventors have also found that when cefdinir:binder ratio is in the range of 5:1 to 1:3, preferably 4:1 to 2:1 in the effervescent formulations comprising cefdinir, the water solubility of cefdinir has increased.

Accordingly, another aspect of the present invention is the effervescent formulations comprising cefdinir wherein the ratio of cefdinir to the binder is in the range of 5:1 to 1:3, preferably in the range of 4:1 to 2:1.

The lubricant that can be used in effervescent formulations of the present invention can be selected from, but not limited to, a group comprising calcium stearate, magnesium stearate, polyethylene glycol, PEG 6000, polyvinyl alcohol, potassium benzoate, sodium benzoate. Preferably, PEG 6000 is used as lubricant in the formulations pertaining to the present invention.

The disintegrant that can be used in effervescent formulations of the present invention can be selected from, but not limited to, a group comprising carboxymethyl cellulose calcium, carboxymethyl cellulose sodium, microcrystalline cellulose, silicon dioxide, croscarmellose sodium, crospovidone, hydroxypropyl cellulose, methyl cellulose, povidone, magnesium aluminum silicate and starch or combinations thereof.

The diluent that can be used in effervescent formulations of the present invention can be selected from, but not limited to, a group comprising calcium carbonate, calcium sulphate, dibasic calcium phosphate, tribasic calcium phosphate, calcium phosphate, calcium sulphate, microcrystalline cellulose, lactose, magnesium carbonate, magnesium oxide, maltodextrin, maltose, mannitol, sodium chloride, sorbitol, starch and xylitol or combinations thereof.

The effervescent acid that is used in effervescent formulations pertaining to the present invention can be selected from organic acids such as citric acid, tartaric acid, malic acid, fumaric acid. Preferably, citric acid is used as effervescent acid.

The effervescent base that is used in effervescent formulations pertaining to the present invention can be selected from sodium hydrogen carbonate, sodium carbonate, potassium carbonate and potassium hydrogen carbonate. Preferably, sodium hydrogen carbonate is used as effervescent base.

In effervescent formulation of the present invention, cefdinir or its pharmaceutically acceptable salts, hydrates, solvates or combinations thereof can be used.

The effervescent formulation of the present invention can comprise 5-30% cefdinir or pharmaceutically acceptable solvates, hydrates, enantiomers, racemates, organic salts, inorganic salts, polymorphs, crystal and amorphous forms of cefdinir and 1-20% organic base; 1-8% binder; 0.1-2% lubricant; 0.1-8% taste regulating agents, 0-25% diluent; 0-15% disintegrant; 0-10% humectant; 0.2-6% coloring agent and/or flavoring agent, 10-60% effervescent acid and 15-50% effervescent base with respect to the total amount of unit dose.

In another aspect, the present invention relates to the processes which can be used for the preparation of effervescent formulations comprising pharmaceutically acceptable excipients in addition to cefdinir as the active agent.

According to this, the process in scope of the present invention comprises the granulation of the active agent cefdinir through conventional wet and/or dry granulation methods present in the prior art or mixing cefdinir and the other excipients through dry blending method and then pulverizing them and optionally compressing them in tablet compressing machine to obtain tablet forms.

According to another aspect of the present invention, the pharmaceutical formulation prepared according to the present invention is used in the manufacture of a medicament so as to be used in upper respiratory infections such as ear, nose, throat, otitis media, sinusitis, tonsillitis, pharyngitis; lower respiratory tract infections such as pyelonephritis, cystitis and urethritis; skin or soft tissue infections such as froncle, pyoderma, impetigo; in the treatment and prophylaxis of gonorrhea and lyme diseases.

### Example 1

### Formulation and Process for the Preparation of Effervescent Tablet

| | % of amount present in unit |
|---|---|
| | dose |
| Cefdinir | 13% |
| Organic Base | 6% |
| Effervescent Acid | 41% |
| Effervescent Base | 28% |
| Binder | 3% |
| *Sodium chloride | 4.5% |
| *Sucralose | 1.5% |
| Lubricant | 2% |
| Coloring Agent | 1% |
| Flavoring Agent | 2% |

*Sodium chloride:sucralose ratio is 3:1.

The formulation to be used in scope of said invention comprises granulating sodium hydrogen carbonate and cefdinir with aqueous organic base solution; then adding citric acid and sodium chloride into the obtained granule and granulating the mixture obtained with binder solution; following this, adding sucralose, lubricant, coloring agent and flavoring agent to the granule mixture obtained and mixing it again; then compressing the obtained mixture in tablet compressing machine to obtain tablets.

### Comparative Example 1

### Formulation and Process for the Preparation of Effervescent Tablet

| | % of amount present in unit |
|---|---|
| | dose |
| Cefdinir | 13% |
| Organic Base | 6% |
| Effervescent Acid | 38% |
| Effervescent Base | 30% |
| Binder | 3.5% |
| *Sodium chloride | 4% |
| *Sucralose | 0.5% |
| Lubricant | 1.5% |
| Coloring Agent | 1.5% |
| Flavoring Agent | 2% |

*Sodium chloride:sucralose ratio is 8:1.

The formulation comprises granulating sodium hydrogen carbonate and cefdinir with aqueous organic base solution; then adding citric acid and sodium chloride into the obtained granule and granulating the mixture obtained with binder solution; following this, adding sucralose, lubricant, coloring agent and flavoring agent to the granule mixture obtained and mixing it again; then compressing the obtained mixture in tablet compressing machine to obtain tablets.

### Comparative Example 2

### Formulation and Process for the Preparation of Effervescent Tablet

| | % of amount present in unit |
|---|---|
| | dose |
| Cefdinir | 15% |
| Organic Base | 6% |
| Effervescent Acid | 38% |
| Effervescent Base | 31% |
| Binder | 3.5% |
| *Sodium chloride | 2.5% |
| Lubricant | 1.5% |
| Coloring Agent | 0.5% |
| Flavoring Agent | 2% |

*Only sodium chloride is used a s taste regulating agent

The formulation comprises granulating sodium hydrogen carbonate and cefdinir with aqueous organic base solution; then adding citric acid and sodium chloride into the obtained granule and granulating the mixture obtained with binder solution; following this, adding lubricant, coloring agent and flavoring agent to the granule mixture obtained and mixing it again; then compressing the obtained mixture in tablet compressing machine to obtain tablets.

### Comparative Example 3

### Formulation and Process for the Preparation of Effervescent Tablet

| | % of amount present in unit |
|---|---|
| | dose |
| Cefdinir | 12% |
| Organic Base | 6% |
| Effervescent Acid | 41.5% |
| Effervescent Base | 32% |
| Binder | 3.0% |
| *Sucralose | 1.5% |
| Lubricant | 1.5% |
| Coloring Agent | 0.5% |
| Flavoring Agent | 2% |

*Only sucralose is used as taste regulating agent

The formulation comprises granulating sodium hydrogen carbonate and cefdinir with aqueous organic base solution; then adding citric acid into the obtained granule and granulating the mixture obtained with binder solution; following this, adding sucralose, lubricant, coloring agent and flavoring agent to the granule mixture obtained and mixing it again; then compressing the obtained mixture in tablet compressing machine to obtain tablets.

### Results:

The inventors have studied on the dissolution data and bitterness values of the compositions illustrated in each example given above. Based on the studies on the comparison of their dissolution data and the bitterness values, the results shown in Tables 1-5 are obtained. Table 1 indicates the comparative data of dissolution time of the compositions for each example. In Table 2 to Table 5, the results of the bitterness values of each cefdinir compositions which are calculated according to the European Pharmacopoeia 5.0 (2.8.15, pg. 221) are shown.

TABLE 1
Comparative Data of Dissolution of Effervescent
Tablets Comprising Cefdinir

| | Dissolution Time (′) |
|---|---|
| Sample | minute (″) second |
| Composition of Example 1 | 2′25″ |
| Composition of Comparative Example 1 | 4′25″ |
| Composition of Comparative Example 2 | 5′32″ |
| Composition of Comparative Example 3 | 6′11″ |

The inventors have observed that the optimum dissolution time is less than 5 minutes for the effervescent formulations comprising cefdinir.

The results of the dissolution data of compositions of Example 1, Comparative example 1, Comparative example 2, Comparative example 3 clearly indicate that the effervescent tablet of the present invention in which sodium chloride and sucralose are used as two different taste regulating agent and the ratio of sodium chloride to sucralose is in the range of 5:1 to 1:1, has a dissolution time that is less than 5 minutes. This shows that the effervescent tablets prepared according to the present invention is more preferable than the other tablets via their higher dissolution rate.

TABLE 2
Results of the Taste Trial for Cefdinir Composition of Example 1

| Panelist* | k* | Y* | X* | Bitterness Value* |
|---|---|---|---|---|
| 1 | 0.88 | 100 | 8 | 110 |
| 2 | 0.72 | 100 | 6 | 120 |
| 3 | 0.8 | 10000 | 3 | 26667 |
| 4 | 0.84 | 100 | 3 | 280 |
| 5 | 0.72 | 10000 | 8 | 9000 |
| 6 | 0.76 | 50000 | 3 | 126667 |
| Average | | | | 27141 |

Panelist*: a member of a taste panel of six people; k*: the correction factor for each panelist; Y*: Dilution Factor (DF) of solution D which is a diluted solution having still a bitter test; X*: number of milliliters of solution D which, when diluted to 10 ml with water, still has a bitter taste; Bitterness value: a value calculated by using the expression of
$\frac{Y \times k}{X \times 0.1}$

TABLE 3
Results of the Taste Trial for Cefdinir Composition of
Comparative Example 1

| | | | | Bitterness |
|---|---|---|---|---|
| Panelist* | k* | Y* | X* | Value* |
| 1 | 0.88 | 100 | 3 | 267 |
| 2 | 0.72 | 10000 | 6 | 12000 |
| 3 | 0.8 | 10000 | 6 | 13333 |
| 4 | 0.84 | 50000 | 2 | 210000 |
| 5 | 0.72 | 50000 | 3 | 120000 |
| 6 | 0.76 | 10000 | 2 | 38000 |
| Average | | | | 43600 |

TABLE 4
Results of the Taste Trial for Cefdinir Composition of
Comparative Example 2

| | | | | Bitterness |
|---|---|---|---|---|
| Panelist* | k* | Y* | X* | Value* |
| 1 | 0.88 | 50000 | 3 | 146667 |
| 2 | 0.72 | 10000 | 3 | 24000 |
| 3 | 0.8 | 50000 | 3 | 133334 |
| 4 | 0.84 | 10000 | 3 | 28000 |
| 5 | 0.72 | 10000 | 2 | 36000 |
| 6 | 0.76 | 10000 | 1.5 | 50667 |
| Average | | | | 69778 |

TABLE 5
Results of the Taste Trial for Cefdinir Composition of
Comparative Example 3

| | | | | Bitterness |
|---|---|---|---|---|
| Panelist* | k* | Y* | X* | Value* |
| 1 | 0.88 | 10000 | 2 | 44000 |
| 2 | 0.72 | 10000 | 3 | 24000 |
| 3 | 0.8 | 50000 | 3 | 133334 |
| 4 | 0.84 | 10000 | 2 | 42000 |
| 5 | 0.72 | 100000 | 3 | 240000 |
| 6 | 0.76 | 10000 | 1.5 | 50667 |
| Average | | | | 89000 |

Taste trials were performed by a taste panel comprising 6 persons. The average bitterness value of each effervescent composition of Example 1, Comparative example 1, Comparative example 2, Comparative example 3 are calculated according to the European Pharmacopoeia 5.0. Six panelists tasted the solutions of compositions prepared according to the method described in European Pharmacopoeia 5.0. The results are represented in Tables 2-5 and they clearly indicate that average bitterness value of the composition of example 1 is the lowest one. Therefore, it can be deduced that effervescent tablets formulated according to the present invention in which sodium chloride and sucralose are used as two different taste regulating agent and the ratio of sodium chloride to sucralose is in the range of 5:1 to 1:1 shown in example 1 is much preferred via its more pleasant taste.

### Formulations Comprising Ceftibuten

The subject of the present invention also relates to the effervescent formulations comprising ceftibuten and/or its pharmaceutically acceptable salts, hydrates, solvates, esters, amorphous and crystal forms and/or a combination thereof and the procedures for their preparation. Surprisingly, it has been found that the effervescent formulations comprising ceftibuten in which at least one non-cellulose based binder having an average particle size in the range of 600-150 μm is used and the ratio of ceftibuten to said binder is in the range of 7:1 to 4:1, has an optimum hardness and disintegrate quickly in water, and provide an effective administration during use.

According to this, the first aspect of the present invention is effervescent formulations comprising ceftibuten in which at least one non-cellulose based binder having an average particle size in the range of 60-150 μm is used and the ratio of ceftibuten to said binder is in the range of 7:1 to 4:1.

Based on the studies of the inventors, in order to obtain ceftibuten effervescent tablets which have desirable hardness and are convenient for the effective administration, it is found that the optimum hardness of the effervescent tablets is in the range of 6-8 kP and the optimum dissolution time is maximum 5 minutes.

According to this, another aspect of the present invention is that the ceftibuten effervescent forms, in which at least one non-cellulose based binder having an average particle size of 60-150 μm is used and the ratio of ceftibuten to binder is in the range of 7:1 to 4:1, have a hardness value in the range of 6-8 kP and dissolve in water within 5 minutes.

Ceftibuten can be present in the form of its solvates, monohydrates, dihydrates, trihydrates, esters, enantiomers, racemates, organic salts, inorganic salts, polymorphs, crystal and amorphous forms or in free form and/or a combination thereof in the effervescent formulations. Preferably, ceftibuten dihydrate and/or trihydrate is used in scope of the present invention.

The binder that can be used in the effervescent formulations pertaining to the present invention can be selected from, but not limited to, a group comprising magnesium aluminum silicate, gelatin, chitosan, polyethylene oxide, guar gum, starch, povidone or combinations thereof. Preferably povidone is used in scope of the present invention.

According to these, another aspect of the present invention is effervescent formulations comprising ceftibuten dihydrate and/or trihydrate in which povidone as a binder having an average particle size in the range of 60-150 μm is used and the ratio of ceftibuten to povidone is in the range of 7:1 to 4:1.

The inventors have found that using the non-cellulose based binder in the range of 1-5%, preferably in the range of 1.5-4%, most preferably in the range of 2-3% with respect to total weight of the unit dose has a positive effect on the disintegration of the effervescent formulations comprising ceftibuten in water.

According to this, another aspect of the present invention is effervescent formulations comprising seftibuten which contain the binder in the range of 0.5-5%, preferably in the range of 1-4%, most preferably in the range of 2-3% of the unit dose weight.

Another aspect of the present invention is effervescent formulations comprising ceftibuten as the active agent, at least one non-cellulose-based binder and pharmaceutically acceptable excipients.

Various excipients selected from, but not limited to, a group comprising lubricants, disintegrants, diluent, taste regulating agents, effervescent acid and effervescent base can be used in the effervescent formulations.

The lubricant that can be used in the effervescent formulations pertaining to the present invention can be selected from, but not limited to, a group comprising calcium stearate, magnesium stearate, polyethylene glycol, PEG 6000, polyvinyl alcohol, potassium benzoate, sodium benzoate. Preferably, PEG 6000 is used as the lubricant in scope of the present invention.

The disintegrant that can be used in effervescent formulations pertaining to the present invention can be selected from, but not limited to, a group comprising carboxymethyl cellulose calcium, carboxymethyl cellulose sodium, microcrystalline cellulose, silicon dioxide, croscarmellose sodium, crospovidone, hydroxypropyl cellulose, methyl cellulose, povidone, magnesium aluminum silicate and starch or combinations thereof.

The diluent that can be used in effervescent formulations pertaining to the present invention can be selected from, but not limited to, a group comprising calcium carbonate, calcium sulphate, dibasic calcium phosphate, tribasic calcium phosphate, calcium phosphate, calcium sulphate, microcrystalline cellulose, lactose, magnesium carbonate, magnesium oxide, maltodextrin, maltose, mannitol, sodium chloride, sorbitol, starch and xylitol or combinations thereof.

The taste regulating agent that can be used in effervescent formulations pertaining to the present invention can be selected from, but not limited to, a group comprising acesulfame, aspartame, dextrose, fructose, glucose, lactitol, maltitol, maltose, sorbitol, saccharine, saccharine sodium, saccharose, sodium cyclamate, sucralose, sodium chloride, potassium chloride, sucrose, xylitol or combinations thereof. Preferably, sucrose, aspartame, sodium chloride or a combination thereof is used in the formulations according to the present invention. Preferably, saccharose, aspartame, sodium chloride or a combination thereof is used as the taste regulating agent in the effervescent formulations pertaining to the present invention.

Ceftibuten is a molecule having a bitter taste. Therefore, this problem should be taken into consideration when developing effervescent formulations comprising this molecule.

Therefore, the inventors have found that the bitter taste of the effervescent forms comprising ceftibuten is eliminated and a pleasant taste is achieved in the case that the ratio of the taste regulating agent is used in an amount of 1-8%, preferably 2-6%, most preferably 3-5% with respect to the total unit dose weight.

According to this, another aspect of the present invention is effervescent formulations in which the ratio of the taste regulating agent is 1-8%, preferably 2-6%, most preferably 3-5% with respect to total unit dose weight.

According to another aspect, the ratio of ceftibuten to the taste regulating agent is in the range of 1:1 to 4:1, preferably 2:1 to 3:1 in the effervescent formulations which are the subject of the present invention.

Effervescent acid that is used in effervescent formulations pertaining to the present invention can be selected from organic acids such as citric acid, tartaric acid, malic acid, fumaric acid, ascorbic acid, adipic acid.

Effervescent base that is used in effervescent formulations pertaining to the present invention can be selected from basic agents such as sodium hydrogen carbonate, sodium carbonate, potassium carbonate, potassium bicarbonate, sodium glycine carbonate, lysine carbonate, arginine carbonate and calcium carbonate.

The effervescent formulations pertaining to the present invention may comprise ceftibuten or pharmaceutically acceptable solvates, monohydrate form, dihydrate form, trihydrate form, enantiomers, racemates, organic salts, inorganic salts, polymorphs, crystal and amorphous forms of 5-50% of the total weight of the unit dose.

The effervescent formulation of the present invention can comprise 5-50% ceftibuten; 1-5% binder; 0.1-8% lubricant; 0-15% disintegrant and/or disintegrants; 0-55% diluent; 1-8% taste regulating agent; 0.5-5% flavoring agents and 10-45% effervescent acid and 20-75% effervescent base with respect to the total amount of unit dose.

In another aspect, the present invention relates to the processes which can be used for the preparation of single dose effervescent formulations comprising pharmaceutically acceptable excipients in addition to ceftibuten as the active agent.

The pharmaceutical compositions of the present invention can be prepared through a process which is comprised of dry blending or dry granulation or wet granulation of the components or a combination thereof and generally known standard techniques and manufacture procedures in technology.

In another aspect, the present invention relates to the preparation of a medicament comprising ceftibuten so as to be used in the treatment of infections caused by gram positive and gram negative bacteria.

According to another aspect of the present invention, the pharmaceutical composition prepared according to the present invention is used in the treatment of upper respiratory infections such as ear, nose, throat, otitis media, sinusitis, tonsillitis, pharyngitis; lower respiratory tract infections such as pyelonephritis, cystitis and urethritis; skin or soft tissue infections such as froncle, pyoderma, impetigo; in the treatment and prophylaxis of gonorrhea and lyme diseases.

The effervescent formulations pertaining to the present invention can be prepared as described below, but not limited to these examples.

### Example 2

### Formulation for the Preparation of Effervescent Granules

| | % of amount present in unit dose |
|---|---|
| Ceftibuten dihydrate | 13.0% |
| Effervescent acid | 28.0% |
| Effervescent base | 51.0% |
| Povidone | 2.0% |
| Taste regulating agent | 3.5% |
| Lubricant | 1.5% |
| Aroma | 1.0% |

The pharmaceutical composition pertaining to the present invention is prepared through granulation of effervescent couple and taste regulating agent with a granulation solution comprising povidone and a suitable solvent; drying the obtained granules; and then mixing them with ceftibuten, lubricant and aroma.

### Example 3

### Formulation for the Preparation of Effervescent Tablets

| | % of amount present in unit dose |
|---|---|
| *Ceftibuten dihydrate | 10.0% |
| Effervescent acid | 33.0% |
| Effervescent base | 49.0% |
| *Povidone | 2.5% |
| Taste regulating agent | 3.0% |
| Lubricant | 1.0% |
| Aroma | 1.5% |

*Ceftibuten Dihydrate:povidone ratio is 5:1

The pharmaceutical composition pertaining to the present invention is prepared through granulation of effervescent couple and taste regulating agent with a granulation solution comprising povidone having a particle size of 100 μm and a suitable solvent; drying the obtained granules; then mixing them with ceftibuten, lubricant and aroma; and finally compressing them into tablets.

### Comparative Example 4

### Formulation for the Preparation of Effervescent Tablets

| | % of amount present in unit dose |
|---|---|
| *Ceftibuten dihydrate | 7.5% |
| Effervescent acid | 33.5% |
| Effervescent base | 51.0% |
| *Povidone | 2.5% |
| Taste regulating agent | 3.0% |
| Lubricant | 1.0% |
| Aroma | 1.5% |

*Ceftibuten Dihydrate:povidone ratio is 3:1

The pharmaceutical composition pertaining to the present invention is prepared through granulation of effervescent couple and taste regulating agent with a granulation solution comprising povidone having a particle size of 200 μm and a suitable solvent; drying the obtained granules; then mixing them with ceftibuten, lubricant and aroma; and finally compressing them into tablets.

The inventors have studied on the values of the hardness and dissolution time of the effervescent compositions compressed in the effervescent tablet which are illustrated in Example 3 and Comparative example 4. According to the studies based on the comparison of their hardness and dissolution time values, the results represented in Tables 6 are observed.

TABLE 6
Comparative Data of Hardness and Dissolution Rate of
Ceftibuten Compositions in Effervescent Tablet Form

| | Average | | | |
|---|---|---|---|---|
| | Particle | | | Dissolution |
| | Size of | | | Time |
| | Povidone | Ceftibuten:Povidone | Hardness | (′) minute |
| Sample | (μm) | Ratio | (kP) | (″) second |
| Composition | 100 | 5:1 | 7 | 2′ 38″ |
| of | | | | |
| Example 3 | | | | |
| Composition | 200 | 3:1 | 10 | 5′ 75″ |
| of | | | | |
| Comparative | | | | |
| Example 4 | | | | |

Table 6 indicates the comparative data of hardness and dissolution time of the compositions for each example. The results clearly show that hardness of the composition of example 3 is in the range of 6-8 kP and thus the dissolution time of this example is below 5 minutes. Therefore, it can be concluded that the effervescent tablet of the present invention in which average particle size of povidone is in the range of 60-150 μm and the ratio of ceftibuten to povidone is in the range of 7:1 to 4:1 is much preferred via its optimum hardness and dissolution time.

### Compositions Comprising Cefuroxime Axetil

The present invention also relates to pharmaceutical compositions comprising (Z)-3-Carboxymethyl-7-(2-(2-furyl)-2-methoxyiminoacetylamino)-3-sefem-4-carboxylic acid which is named as cefuroxime axetil or any pharmaceutically acceptable derivative thereof, and the use of these compositions in the treatment of bacterial infections.

Accordingly, the subject of the present invention relates to the suspension formulations comprising cefuroxime axetil and/or hydrates, solvates, esters enantiomers, polymorphs, crystal forms, amorphous forms, salt forms or free base form and/or combinations thereof and the procedures for their preparation. Surprisingly, it has been found that pharmaceutical compositions in suspension form comprising cefuroxime axetil wherein at least two pH agents are used and the ratio of the first pH agent having 46-100% (w/w), preferably 50-80% (w/w), more preferably 55-75% (w/w) water solubility at 25° C. and the second pH agent having 1-45% (w/w), preferably 5-35%, more preferably 10-25% water solubility at 25° C. is in the range of 15:1 and 1:1 by weight, have achieved to overcome the solubility problem of cefuroxime axetil by preventing the gelling and also eliminate the bitter taste of cefuroxime axetil

According to this, the first aspect of the present invention is the pharmaceutical compositions in suspension form comprising cefuroxime axetil in which at least two pH agents are used and the ratio of the first pH agent having 46-100% (w/w), preferably 50-80% (w/w), more preferably 55-75% (w/w) water solubility at 25° C. and the second pH agent having 1-45% (w/w), preferably 5-35%, more preferably 10-25% water solubility at 25° C. is in the range of 15:1 and 1:1 by weight.

The term “pH agent having 46-100% (w/w) water solubility at 25° C.” means that the amount of pH agent dissolved in 100 g water is in the range of 46-100 g at 25° C.

According to this aspect, the inventors have seen that the selection of the pH agent according to its solubility and the presence of more than one pH agent in the formulation provide both the increase of the solubility of cefuroxime axetil and masking the bitter taste of it.

Cefuroxime which is comprised in the suspension formulation pertaining to the present invention can be in the form of its pharmaceutically acceptable hydrates, solvates enantiomers, polymorphs, crystal forms, amorphous forms, and/or combinations thereof.

The first pH agent having 46-100% (w/w) water solubility at 25° C. that is used in the suspension formulation pertaining to the present invention can be selected from a group comprising acetic acid, citric acid, potassium citrate, carbonic acid, hydrochloric acid, monosodium glutamate, lactic acid, malic acid, propionic acid, sulfuric acid, tartaric acid, sodium hydroxide or a combination thereof. Preferably, citric acid is used as the first pH agent.

The second pH agent having 1-45% (w/w) water solubility at 25° C. that is used in the suspension formulation pertaining to the present invention can be selected from sodium acetate, trisodium citrate, phosphoric acid, glutamic acid, dicalcium phosphate, trisodium phosphate, calcium sulfate or a combination thereof. Preferably, trisodium citrate is used as the second pH agent.

According to these, another aspect of the present invention is the pharmaceutical compositions in suspension form comprising cefuroxime axetil in which citric acid and trisodium citrate are used as pH agents and the ratio of citric acid to trisodium citrate is in the range of 15:1 and 1:1 by weight.

The total amount of the pH agents that are used in the suspension formulation pertaining to the present invention is in the range of 1-20%, preferably in the range of 3-9% by weight.

The suspension formulation pertaining to the present invention can comprise one or several of the excipients including binders, lubricants, glidants, viscosity agents, disintegrants, diluents, preservatives, flavoring agents, sweeteners, coloring agents, surfactants, antifoam agents and stabilizing agents in addition to active agent and at least two pH agents.

The binder that is used in the pharmaceutical formulation pertaining to the present invention can be selected from, but not limited to, a group comprising starches such as potato starch, corn starch, wheat starch; sugars such as sucrose, glucose, dextrose, lactose, maltodextrin; natural and synthetic gums; gelatin; cellulose derivatives such as microcrystalline cellulose, HPC, HEC, HPMC, carboxymethyl cellulose, methyl cellulose, ethyl cellulose; polyvinylpyrrolidone (povidone), polyethylene glycol (PEG); waxes; calcium carbonate; calcium phosphate; alcohols such as sorbitol, xylitol, mannitol, and water or a combination thereof.

The lubricant that is used in the pharmaceutical formulation pertaining to the present invention can be selected from, but not limited to, a group comprising talc, magnesium stearate, stearic acid, sodium stearil fumarate, polyoxyethylene glycol, leucine, alanine, glycine, sodium benzoate, sodium acetate, fumaric acid or a combination thereof.

The disintegrant that is used in the pharmaceutical formulation pertaining to the present invention can be selected from, but not limited to, a group comprising starches such as potato starch, corn starch, wheat starch, pregelatinized starch, sodium starch glycolate; cellulose derivatives such as croscarmellose sodium or microcrystalline cellulose; polyvinylpyrrolidone; crospovidone; alginic acid and its salts; clays such as xanthan gum or Veegum; ion exchange resins or a combination thereof.

The diluent that is used in the pharmaceutical formulation pertaining to the present invention can be selected from, but not limited to, a group comprising lactose, maltose, dextrin, maltodextrin, mannitol, sorbitol, starch and a combination thereof.

The glidant that is be used in the pharmaceutical formulation pertaining to the present invention can be selected from, but not limited to, a group comprising magnesium silicate, silicon dioxide, starch, talc, aerosil 200, tribasic calcium phosphate or a combination thereof.

The flavoring agent that is used in the pharmaceutical formulation pertaining to the present invention can be selected, but not limited to, a group comprising natural aroma oils (peppermint oil, wintergreen oil, clove bud oil, parsley oil, eucalyptus oil, lemon oil, orange oil), menthol, menthane, anethole, methyl salicylate, eucalyptol, cinnamon, 1-methyl acetate, sage, eugenol, oxanone, alpha irisone, marjoram, lemon, orange, blackberry, propenyl guaetol acetyl, cinnamon, vanilla, thymol, linalol, cinnamaldehyde glycerol acetal, N-substituted p-menthane-3-carboxamide, 3,1-methoxy propane 1,2-diol or a combination thereof.

The preservative that is used in the pharmaceutical formulation pertaining to the present invention can be selected, but not limited to, a group comprising propylene glycol, disodium edentate, benzalkonium chloride, benzoic acid, butyl paraben, cetrimide, chlorobutanol, phenyl mercuric acetate, potassium sorbate, sodium benzoate, sorbic acid, methyl paraben or a combination thereof. Preferably, sodium benzoate is used as preservative.

The sweetener that is used in the pharmaceutical formulation pertaining to the present invention can be selected from, but not limited to, a group comprising sucralose, sucrose, fructose, glucose, galactose, xylose, dextrose, laevulose, lactose, maltose, maltodextrin, mannitol, maltitol, maltol, sorbitol, xylitol, erythritol, lactitol, isomalt, corn syrup, saccharine, saccharine salts, acesulfame potassium, aspartame, D-tryptophane, monoammonium glycyrrhizinate, neohesperidin dihydrochalcone, thaumatin, neotame, alitame, stevioside and cyclamates or a combination thereof.

The viscosity agent that is used in the pharmaceutical formulation pertaining to the present invention can be selected from a group comprising carboxymethyl cellulose, methyl cellulose, xanthan gum, gummi tragacanthae, gum arabic, collidone, agar-agar, bentonite, hydroxyethyl cellulose, or a combination thereof.

The inventors have observed that the amount of the viscosity agent used in the formulation is a parameter that has an influence on the solubility of cefuroxime axetil by decreasing the formulation of gelatinous mass. In the case that the viscosity agent is used in an amount of 0.1-4% by weight in the formulation, the increase in the solubility of cefuroxime axetil can be achieved to some extent.

Accordingly, the present invention relates to the pharmaceutical compositions comprising the viscosity agent in an amount of 0.1-4% by weight.

According to the present invention, the formulation in suspension form pertaining to the present invention comprises;
Cefuroxime axetil in the range of 1-30% by weight
pH agent in the range of 1-20% by weight
Viscosity agent in the range of 0.1-4% by weight
Lubricant in the range of 0-5% by weight
Sweetener in the range of 20-90% by weight
Flavoring agent in the range of %0.5-5 by weight
Glidant in the range of %0.1-3 by weight
Preservative in the range of %0.05-4 by weight

According to another aspect, the present invention relates to the use of the pharmaceutical compositions in suspension form comprising cefuroxime axetil and at least two pH agents in the treatment of upper respiratory infections such as ear, nose, throat, otitis media, sinusitis, tonsillitis, pharyngitis; lower respiratory tract infections such as pyelonephritis, cystitis and urethritis; skin or soft tissue infections such as froncle, pyoderma, impetigo in the treatment and prophylaxis of gonorrhea and lyme diseases.

The suspension formulation pertaining to the present invention corresponds to powders or granules for the preparation of suspensions and/or dispersed forms of these powders and granules directly with liquid.

According to another aspect, the present invention relates to the process for the preparation of the suspension formulations comprising cefuroxime axetil pertaining to the present invention comprising the steps of mixing cefuroxime axetil, pH agents, sweetener, viscosity agent, preservative, lubricant, glidant and flavoring agent; sieving them and finally filling the mixture into glass bottles.

### Example 4

| Component name | Amount (% by weight) |
|---|---|
| Cefuroxime axetil | 8% |
| *Citric acid | 3% |
| *Trisodium citrate | 0.3% |
| Preservative | 0.2% |
| Viscosity agent | 1% |
| Sweetener | 84% |
| Glidant | 0.5% |
| Lubricant | 1% |
| Flavoring agent | 2% |

*Citric acid:trisodium citrate ratio is 10:1.

Cefuroxime axetil, citric acid, trisodium citrate, sweetener, viscosity agent, preservative, lubricant, glidant and flavoring agent are mixed; then the mixture is sieved and finally the mixture is filled into glass bottles.

### Comparative Example 5

| Component name | Amount (% by weight) |
|---|---|
| Cefuroxime axetil | 6.5% |
| *Citric acid | 6% |
| *Trisodium citrate | 0.3% |
| Preservative | 0.2% |
| Viscosity agent | 0.5% |
| Sweetener | 84% |
| Glidant | 1% |
| Lubricant | 0.5% |
| Flavoring agent | 1.0% |

*Citric acid:trisodium citrate ratio is 20:1.

Cefuroxime axetil, citric acid, trisodium citrate, sweetener, viscosity agent, preservative, lubricant, glidant and flavoring agent are mixed; then the mixture is sieved and finally the mixture is filled into glass bottles.

### Comparative Example 6

| Component name | Amount (% by weight) |
|---|---|
| Cefuroxime axetil | 7.5% |
| Citric acid | 5% |
| Preservative | 0.2% |
| Viscosity agent | 1% |
| Sweetener | 84% |
| Glidant | 1% |
| Lubricant | 0.3% |
| Flavoring agent | 1.0% |

Cefuroxime axetil, citric acid, sweetener, viscosity agent, preservative, lubricant, glidant and flavoring agent are mixed; then the mixture is sieved and finally the mixture is filled into glass bottles.

### Comparative Example 7

| Component name | Amount (% by weight) |
|---|---|
| Cefuroxime axetil | 7% |
| Trisodium citrate | 4.5% |
| Preservative | 0.7% |
| Viscosity agent | 0.5% |
| Sweetener | 84% |
| Glidant | 1% |
| Lubricant | 0.8% |
| Flavoring agent | 1.5% |

Cefuroxime axetil, trisodium citrate, sweetener, viscosity agent, preservative, lubricant, glidant and flavoring agent are mixed; then the mixture is sieved and finally the mixture is filled into glass bottles.

### Results:

The inventors have studied on the dissolution data and bitterness values of the compositions illustrated in each example given above. According to the studies based on the comparison of their dissolution data and the bitterness values, the results represented in Tables 7-11 are observed. Table 7 indicates the comparative data of dissolution of the compositions for each example. In Table 8 to Table 11, the results of the bitterness values of each cefuroxime axetil compositions which are calculated according to the European Pharmacopoeia 5.0 (2.8.15, pg. 221) are shown.

TABLE 7
Comparative Data of Dissolution of Cefuroxime Axetil
Compositions in Suspension Form

| | | % dissolved |
|---|---|---|
| | | amount of |
| Sample | Time (min) | composition |
| Composition of Example 4 | 45 | 95% |
| Composition of Comparative Example 5 | 45 | 75% |
| Composition of Comparative Example 6 | 45 | 65% |
| Composition of Comparative Example 7 | 45 | 60% |

The results of the dissolution data of compositions of Example 4, Comparative example 5, Comparative example 6, Comparative example 7 clearly indicate that the suspension of the present invention in which citric acid and trisodium citrate are used and citric acid:trisodium ratio by weight is 10:1, as shown in example 4, has a higher dissolution rate compared to the others.

TABLE 8
Results of the Taste Trial for
Cefuroxime Axetil Composition of Example 4

| Panelist* | k* | Y* | X* | Bitterness Value* |
|---|---|---|---|---|
| 1 | 0.76 | 10000 | 6 | 12667 |
| 2 | 0.84 | 100 | 8 | 105 |
| 3 | 0.72 | 10000 | 8 | 9000 |
| 4 | 0.8 | 50000 | 3 | 133335 |
| 5 | 0.76 | 100 | 6 | 127 |
| 6 | 0.72 | 10000 | 8 | 9000 |
| Average | | | | 27372 |

Panelist*: a member of a taste panel of six people; k*: the correction factor for each panelist; Y*: Dilution Factor (DF) of solution D which is a diluted solution having still a bitter test; X*: number of mililitres of solution D which, when diluted to 10 ml with water, still has a bitter taste; Bitterness value: a value computed by using the expression of
$\frac{Y \times k}{X \times 0.1}$

TABLE 9
Results of the Taste Trial for Cefuroxime Axetil
Composition of Comparative Example 5

| | | | | Bitterness |
|---|---|---|---|---|
| Panelist* | k* | Y* | X* | Value* |
| 1 | 0.76 | 10000 | 6 | 12667 |
| 2 | 0.84 | 50000 | 3 | 140000 |
| 3 | 0.72 | 100 | 3 | 240 |
| 4 | 0.8 | 100 | 2 | 400 |
| 5 | 0.76 | 10000 | 3 | 25334 |
| 6 | 0.72 | 50000 | 3 | 120000 |
| Average | | | | 49773 |

TABLE 10
Results of the Taste Trial for Cefuroxime Axetil
Composition of Comparative Example 6

| | | | | Bitterness |
|---|---|---|---|---|
| Panelist* | k* | Y* | X* | Value* |
| 1 | 0.76 | 10000 | 2 | 38000 |
| 2 | 0.84 | 10000 | 1.5 | 56000 |
| 3 | 0.72 | 100000 | 3 | 240000 |
| 4 | 0.8 | 10000 | 1.5 | 53334 |
| 5 | 0.76 | 10000 | 1.5 | 50667 |
| 6 | 0.72 | 10000 | 2 | 36000 |
| Average | | | | 79000 |

TABLE 11
Results of the Taste Trial for Cefuroxime Axetil
Composition of Comparative Example 7

| | | | | Bitterness |
|---|---|---|---|---|
| Panelist* | k* | Y* | X* | Value* |
| 1 | 0.76 | 10000 | 2 | 38000 |
| 2 | 0.84 | 100000 | 3 | 280000 |
| 3 | 0.72 | 10000 | 1.5 | 48000 |
| 4 | 0.8 | 10000 | 1.2 | 66667 |
| 5 | 0.76 | 10000 | 1.5 | 50667 |
| 6 | 0.72 | 10000 | 2 | 36000 |
| Average | | | | 86556 |

Taste trials were performed by a taste panel comprising 6 persons. The average bitterness value of each suspension of the compositions of Example 4, Comparative example 5, Comparative example 6, Comparative example 7 are calculated according to the European Pharmacopoeia 5.0. Six panelists assessed these compositions which are represented in Tables 8-11. The results clearly show that average bitterness value of the composition of example 4 is the lowest one. Therefore, it can be concluded that suspension of the present invention in which citric acid and trisodium citrate are used and citric acid:trisodium ratio by weight is 10:1 shown in example 4 is much preferred for its low bitterness value.

### Formulations Comprising Third Generation Cephalosporins and Clavulanic Acid

The subject of the present invention also relates to water soluble pharmaceutical formulations comprising the third generation cephalosporins and clavulanic acid and/or derivatives thereof. Surprisingly, it was found that formulations comprising the combination of a third generation cephalosporin and clavulanic acid and/or derivatives thereof entirely dissolve in water when a third generation cephalosporin antibiotic is used in 2%-15% of the unit dose; two different pH agents wherein one pH agent has pKa in the range of 1-5, preferably 2-4 and the other has pKa in the range of 5.1-15, preferably 5.5-12 are used to form a buffer system and the ratio of the first pH agent having pKa value of 1-5, preferably 2-4 to the second pH agent having the pKa value of 5.1-15, preferably 5.5-12 is in the range of 3:1 to 1:1.

The inventors have found that the formulations comprising the combination of a third generation cephalosporin and clavulanic acid and/or derivatives thereof according to the present invention have solved both the solubility problem of cefdinir and the stability problem of clavulanic acid and thereby a stable water soluble formulations comprising a combination of a third generation cephalosporin and clavulanic acid have been obtained.

The first aspect of the present invention is the water soluble formulations comprising a third generation cephalosporin and clavulanic acid and/or derivatives thereof in which a third generation cephalosporin antibiotic is used in 2%-15% of the unit dose; two different pH agents wherein one pH agent has pKa in the range of 1-5, preferably 2-4 and the other has pKa in the range of 5.1-15, preferably 5.5-12 are used to form a buffer solution and the ratio of the first pH agent having pKa value of 1-5 to the second pH agent having the pKa value of 5.1-15 is in the range of 3:1 to 1:1.

The water soluble formulations according to present invention can be in the form of powder, granule or tablet.

The third generation cephalosporin that can be used in water soluble formulations of the present invention is used in 2%-15%, preferably 2%-12%, more preferably 3%-10% of the unit dose.

The third generation cephalosporin that can be used in water soluble formulations of the present invention can be selected from the group comprising cefpodoxime, cefditoren, ceftibuten, cefdinir, cefixime, cefetamet.

Preferably, ceftibuten or cefdinir is used in scope of the present invention.

The third generation cephalosporin that can be used in water soluble formulations of the present invention can be in the form of its solvates, hydrates, esters, enantiomers, racemates, organic salts, inorganic salts, polymorphs, in crystalline and amorphous forms or in free form and/or combinations thereof.

In the case that cefpodoxime is used in the present invention it is preferably used in its ester form, more preferably in cefpodoxime proxetil form.

In the case that cefditoren is used in the present invention it is preferably used in its ester form, more preferably in cefditoren pivoxil form.

In the case that cefixime is used in the present invention it is preferably used in its hydrate form, more preferably in trihydrate form.

In the case that ceftibuten is used in the present invention it is preferably used in its hydrate form, more preferably in dihydrate and/or trihydrate form.

In the case that cefdinir is used in the present invention it is preferably used in free form.

Clavulanic acid that can be used in the pharmaceutical composition of the present invention can be in the form of its solvates, hydrates, enantiomers, racemates, organic salts, alkaline metal salts, alkaline earth metal salts, inorganic salts, polymorphs, in crystalline and amorphous forms or in free form and/or combinations thereof.

Preferably, potassium clavulanate is used in the present invention.

The first pH agent, which has pKa in the range of 1-5, preferably 2-4, to be used in the formulation of the present invention can be selected from, but not limited to, citric acid and malic acid or a combination thereof. Preferably, citric acid is used.

The second pH agent, which has pKa in the range of 5.1-15, preferably 5.5-12, to be used in the formulation of the present invention can be selected from, tribasic calcium phosphate, monosodium glutamate, potassium citrate, trisodium citrate, sodium hydroxide, dibasic sodium phosphate, monobasic sodium phosphate or combinations thereof. Preferably, trisodium citrate is used.

Another aspect of the present invention, the water soluble formulations comprising cefdinir or ceftibuten and clavulanate in which cefdinir or ceftibuten is used in 2%-15% of the unit dose; citric acid and trisodium citrate are used to form a buffer system and the ratio of citric acid to trisodium citrate is in the range of 3:1 to 1:1.

Another aspect of the present invention is water soluble formulations comprising pharmaceutically acceptable excipients in addition to the combination of the third generation cephalosporins and clavulanic acid and/or derivatives thereof as the active agents and the pH agents.

In the formulation of the present invention, various excipients selected from, but not limited to, the group comprising sweeteners, preservative agents, viscosity agents, glidants, lubricants, disintegrants, diluents and flavoring agents can be used in addition to the third generation cephalosporin, clavulanic acid and pH agents.

The sweetener to be used in the formulation of the present invention can be selected from, but not limited to, a group comprising acesulfame, aspartame, dextrose, fructose, glucose, lactitol, maltitol, sugar, maltose, sorbitol, saccharine, saccharine sodium, saccharose, sodium cyclamate, sucralose, sodium chloride, potassium chloride, sucrose, xylitol or combinations thereof. Preferably, saccharose is used as the sweetener in the present invention.

The preservative agent to be used in the formulation of the present invention can be selected from a group comprising ascorbic acid, citric acid, malic acid, propionic acid, sodium ascorbate, sodium benzoate or combinations thereof. Preferably, sodium benzoate is used as the preservative agent.

The viscosity agent to be used in the formulation of the present invention can be selected from a group comprising carboxymethyl cellulose sodium, carboxymethyl cellulose calcium, chitosan, colloidal silicon dioxide, gelatin, guar gum, xanthan gum, hydroxyethyl cellulose, hydroxypropyl cellulose, hydroxymethyl cellulose, hypromellose, maltodextrin, polyvinyl alcohol or combinations thereof. Preferably, xanthan gum is used in the present invention.

Based on the studies of the inventors, it has been observed that the ratio of the third generation cephalosporin to xanthan gum is an important parameter influencing the dispersion time of the water soluble formulations comprising a third generation cephalosporin and clavulanic acid or derivatives thereof. They have found that the dispersion time of the granules and powders comprising the formulations according to present invention in which the ratio of the third generation cephalosporin to xanthan gum is in the range of 7:1 to 15:1, preferably 8:1 to 13:1, more preferably 9:1 to 12:1 is shorter compared to the formulations comprising xanthan gum less or more than this amount.

According to this, another aspect of the present invention is water soluble formulations comprising a third generation cephalosporin and clavulanic acid or derivatives thereof in which the ratio of the third generation cephalosporin to xanthan gum is in the range of 7:1 to 15:1.

The glidant that can be used in the pharmaceutical combination of the present invention can be selected from, but not limited to, a group comprising magnesium silicate, silicon dioxide, starch, talc, tribasic calcium phosphate or a combination thereof. Preferably, silicon dioxide is used as the glidant.

The lubricant that can be used in the pharmaceutical formulation of the present invention can be selected from, but not limited to, a group comprising calcium stearate, magnesium stearate, polyethylene glycol, PEG 6000, polyvinyl alcohol, potassium benzoate, talc, sodium benzoate or a combination thereof. Preferably, magnesium stearate is used as the lubricant in the pharmaceutical formulation pertaining to the present invention.

The disintegrant that can be used in the pharmaceutical formulation pertaining to the present invention can be selected from, but not limited to, a group comprising carboxymethyl cellulose calcium, carboxymethyl cellulose sodium, microcrystalline cellulose, silicon dioxide, croscarmellose sodium, crospovidone, hydroxypropyl cellulose, methylcellulose, povidone, magnesium aluminium silicate, starch or combinations thereof.

The diluent that can be used in the pharmaceutical formulation of the present invention can be selected from, but not limited to, a group comprising calcium carbonate, calcium sulphate, dibasic calcium phosphate, tribasic calcium phosphate, calcium sulphate, microcrystalline cellulose, lactose, magnesium carbonate, magnesium oxide, maltodextrin, maltose, mannitol, sodium chloride, sorbitol, starch, xylitol or combinations thereof.

In the pharmaceutical formulation of the present invention, 20-700 mg of a third generation cephalosporin or its pharmaceutically acceptable salts, hydrates, solvates or combinations thereof in quantities equal to this can be used.

In the pharmaceutical formulation of the present invention, 50-450 mg of clavulanic acid or its pharmaceutically acceptable salts, hydrates, solvates or combinations thereof in quantities equal to this can be used.

Clavulanic acid and its derivatives (e.g. potassium clavulanate) are extremely sensitive to moisture. Therefore, potassium clavulanate is used together with a humectant in the ratio of 1:1 in the pharmaceutical formulation of the present invention.

The humectant that can be used in the pharmaceutical formulation of the present invention can be selected from silica, colloidal silica, magnesium trisilicate, powdered cellulose, magnesium oxide, calcium silicate, starch, syloid, microcrystalline cellulose and talc. Preferably, syloid or microcrystalline cellulose is used as humectants.

In the pharmaceutical formulation of the present invention, potassium clavulanate is used with syloid or microcrystalline cellulose preferably in the ratio of 1:1.

The amount of the third generation cephalosporin or its pharmaceutically acceptable solvates, hydrates, enantiomers, racemates, organic salts, inorganic salts, polymorphs, crystalline or amorphous forms that the pharmaceutical formulation of the present invention can comprise is in the range of 2% to 15% with respect to the total weight of the unit dose.

The pharmaceutical formulation of the present invention can comprise clavulanic acid in the range of 5% to 30% with respect to the total weight of the unit dose or its pharmaceutically acceptable salts, hydrates, solvates or combinations thereof in quantities equal to this.

The pharmaceutical formulation of the present invention can comprise 2-15% a third generation cephalosporin, 5-30% potassium clavulanate, 0.1-4% glidant, 0.2-3% lubricant, 0-20% disintegrant and/or disintegrants, 0-15% diluent, 30-90% sweetener, 0.5-4% pH agent, 0.1-2% preservative agent, 0.2-4% viscosity agent and 0.1-5% flavoring agent by weight with respect to the total weight of the unit dose.

In another aspect, the present invention relates to processes that can be used for preparation of the pharmaceutical formulations comprising pharmaceutically acceptable excipients in addition to the third generation cephalosporin and clavulanic acid or derivatives thereof as the active agent.

According to this, the process in scope of the present invention comprises granulation of the active agents, in other words third generation cephalosporin and clavulanic acid or derivatives thereof by conventional wet and/or dry granulation methods; or mixing the third generation cephalosporin, clavulanic acid derivatives and the other excipients by dry blending method and then pulverizing them, optionally compressing the obtained mixture in tablet form.

Another aspect of the present invention is the use of the pharmaceutical formulation prepared according to the present invention in the treatment of diseases related to infection caused by gram positive and gram negative bacteria.

Another aspect of the present invention, on the other hand, is the use of the pharmaceutical formulation prepared according to the present invention in the production of a medicament so as to be used in upper respiratory infections such as ear, nose, throat, otitis media, sinusitis, tonsillitis, pharyngitis; lower respiratory tract infections such as pyelonephritis, cystitis and urethritis; skin or soft tissue infections such as froncle, pyoderma, impetigo; in the treatment and prophylaxis of gonorrhea and lyme diseases.

The pharmaceutical formulation pertaining to the present invention can be prepared as described below, but not limited to the examples given.

### Example 5

### Formulation and Process for Preparation of Water Soluble Granule Comprising Ceftibuten and Potassium Clavulanate

| | % of amount in unit dose |
|---|---|
| Ceftibuten dihydrate | 5.5% |
| Potassium clavulanate: syloid | 6.0% |
| Sweetener | 83.0% |
| *Citric acid | 1.0% |
| *Trisodium citrate | 0.75% |
| Viscosity agent | 1.0% |
| Lubricant | 0.75% |
| Glidant | 0.5% |
| Preservative agent | 0.5% |
| Flavoring agent | 1.0% |

*Citric acid:trisodium citrate is in the range of 3:1 to 1:1, specifically 1.3:1.

Ceftibuten, potassium clavulanate:syloid, sweetener, viscosity agent, citric acid, trisodium citrate, glidant, flavoring agent and the preservative agent are sieved, then mixed. The lubricant is added into the obtained mixture and the powder mixture is filled into sachets.

### Example 6

### Formulation and Process for Preparation of Water Soluble Granule Comprising Cefdinir and Potassium Clavulanate

| | % of amount in unit dose |
|---|---|
| Cefdinir | 4.5% |
| Potassium clavulanate: | 8.0% |
| microcrystalline cellulose | |
| Sweetener | 81.0% |
| *Citric acid | 1.0% |
| *Trisodium citrate | 0.5% |
| Viscosity agent | 1.0% |
| Lubricant | 1.5% |
| Glidant | 0.5% |
| Preservative agent | 1.0% |
| Flavoring agent | 1.0% |

*Citric acid:trisodium citrate is in the range of 3:1 to 1:1, specifically 2:1.

Cefdinir, potassium clavulanate:microcrystalline cellulose, sweetener, viscosity agent, citric acid, trisodium citrate, glidant, flavoring agent and the preservative agent are sieved, and then mixed. The lubricant is added into the mixture obtained and the powder mixture is filled into sachets.

### Comparative Example 8

### Formulation and Process for Preparation of Water Soluble Granule Comprising Cefdinir and Potassium Clavulanate

| | % of amount in unit dose |
|---|---|
| Cefdinir | 7.0% |
| Potassium clavulanate: | 6.5% |
| microcrystalline cellulose | |
| Sweetener | 80.0% |
| *Citric acid | 2.5% |
| *Trisodium citrate | 0.5% |
| Viscosity agent | 1.0% |
| Lubricant | 1.0% |
| Glidant | 0.5% |
| Preservative agent | 0.5% |
| Flavoring agent | 0.5% |

*Citric acid:trisodium citrate is 5:1

Cefdinir, potassium clavulanate:microcrystalline cellulose, sweetener, viscosity agent, citric acid, trisodium citrate, glidant, flavoring agent and the preservative agent are sieved, and then mixed. The lubricant is added into the mixture obtained and the powder mixture is filled into sachets.

### Comparative Example 9

### Formulation and Process for Preparation of Water Soluble Granule Comprising Cefdinir and Potassium Clavulanate

| | % of amount in unit dose |
|---|---|
| Cefdinir | 6.0% |
| Potassium clavulanate: | 4.5% |
| microcrystalline cellulose | |
| Sweetener | 83.5% |
| *Citric acid | 2.0% |
| Viscosity agent | 1.0% |
| Lubricant | 1.0% |
| Glidant | 0.5% |
| Preservative agent | 0.75% |
| Flavoring agent | 0.75% |

*Only citric acid is used

Cefdinir, potassium clavulanate:microcrystalline cellulose, sweetener, viscosity agent, citric acid, glidant, flavoring agent and the preservative agent are sieved, and then mixed. The lubricant is added into the mixture obtained and the powder mixture is filled into sachets.

### Comparative Example 10

### Formulation and Process for Preparation of Water Soluble Granule Comprising Cefdinir and Potassium Clavulanate

| | % of amount in unit dose |
|---|---|
| Cefdinir | 4.0% |
| Potassium clavulanate: | 5.5% |
| microcrystalline cellulose | |
| Sweetener | 86.0% |
| *Trisodium citrate | 1.5% |
| Viscosity agent | 0.5% |
| Lubricant | 0.75% |
| Glidant | 0.25% |
| Preservative agent | 1.0% |
| Flavoring agent | 0.5% |

*Only trisodium citrate is used

Cefdinir, potassium clavulanate:microcrystalline cellulose, sweetener, viscosity agent, trisodium citrate, glidant, flavoring agent and the preservative agent are sieved, and then mixed. The lubricant is added into the mixture obtained and the powder mixture is filled into sachets.

### Results:

The inventors have studied on the dissolution data of the formulations illustrated in each example given above. According to the studies based on the comparison of their dissolution data, the results represented in Table 12 are observed. Table 12 indicates the comparative data of dissolution of the formulations for each example.

TABLE 12
Comparative Data of Dissolution of Cefdinir/Ceftibuten
Formulations in Water Soluble Form

| | | % dissolved |
|---|---|---|
| | | amount of |
| Sample | Time (min) | formulation |
| Formulation of Example 5 | 45 | 96% |
| Formulation of Example 6 | 45 | 95% |
| Formulation of Comparative Example 8 | 45 | 90% |
| Formulation of Comparative Example 9 | 45 | 82% |
| Formulation of Comparative Example 10 | 45 | 78% |

The results of the dissolution data of formulations of Example 5, Example 6, Comparative example 8, Comparative example 9, Comparative example 10 clearly indicate that the formulations comprising cefdinir or ceftibuten and potassium clavulanate of the present invention in which citric acid and trisodium citrate are used and citric acid:trisodium citrate ratio by weight is in the range of 3:1 to 1:1, as shown in example 5 and example 6, have higher dissolution rate values compared to the others.

What is claimed is:

1. A pharmaceutical formulation formulated in effervescent form comprising cefdinir as the active agent characterized in that two different taste regulating agents are used and the ratio of the first taste regulating agent having 31-90% (w/w) water solubility at 25° C. and the second taste regulating agent having 5-30% (w/w) water solubility at 25° C. is in the range of 5:1 and 1:1.

2. (canceled)

3. The pharmaceutical formulation according to claim 1, wherein the first taste regulating agent used in effervescent formulations of the present invention is selected from a group comprising dextrose, fructose, glucose, lactitol, maltitol, maltose, sorbitol, saccharine sodium, sodium cyclamate, sodium chloride, potassium chloride, sucrose and xylitol or combinations thereof.

4. The pharmaceutical formulation according to claim 3, wherein sodium chloride is used as the first taste regulating agent.

5. The pharmaceutical formulation according to claim 1, wherein the second taste regulating agent used in effervescent formulations of the present invention is selected from a group comprising acesulfame, aspartame, saccharine and sucralose, or combinations thereof.

6. The pharmaceutical formulation according to claim 5, wherein sucralose is used as the second taste regulating agent.

7. The pharmaceutical formulation according to claim 1, wherein the ratio of cefdinir to the combination of taste regulating agents is in the range of 10:1 to 1:1.

8. The pharmaceutical formulation according to claim 1, wherein organic base is used in addition to cefdinir and the taste regulating agents and the ratio of cefdinir to the organic base is in the range of 5:1 to 1:5.

9. The pharmaceutical formulation according to claim 8, wherein primary amines, secondary amines, tertiary amines and/or heterocyclic compounds containing nitrogen can be used as the organic base.

10. The pharmaceutical formulation according to claim 9, wherein organic base that is to be used in the formulation is selected from a group comprising ethanolamine, isopropanolamine, 1-dioxy-1-methylamino-sorbitol, 1-dioxy-1-methylamino-D-glucitol, tris(hydroxymethyl)aminomethane, N-(Tri(hydroxymethyl)methyl)glycine, N,N-Bis(2-hydroxyethyl)glycine, 2-methyl aminophenol.

11. (canceled)

12. The pharmaceutical formulation according to claim 1, further comprising pharmaceutically acceptable excipients, wherein said pharmaceutically acceptable excipients is selected from the group consisting of binders, lubricants, humectants, disintegrants, diluents, and effervescent couple in addition to cefdinir, combination of taste regulating agents, and organic base.

13-14. (canceled)

15. The pharmaceutical formulation according to claim 12, wherein said formulation comprises 1-8% binder.

16. The pharmaceutical formulation according to claim 12, wherein cefdinir:binder ratio is in the range of 5:1 to 1:3.

17-20. (canceled)

21. The pharmaceutical formulation according to claim 12, wherein the effervescent acid is selected from organic acids such as citric acid, tartaric acid, malic acid, fumaric acid.

22. The pharmaceutical formulation according to claim 12, wherein the effervescent base is selected from basic agents such as sodium hydrogen carbonate, sodium carbonate, potassium carbonate and potassium hydrogen carbonate.

23. The pharmaceutical formulation according to claim 12, wherein said formulation comprises 5-30% cefdinir or pharmaceutically acceptable solvates, hydrates, enantiomers, racemates, organic salts, inorganic salts, polymorphs, crystal and amorphous forms of cefdinir and 1-20% organic base; 1-8% binder; 0.1-2% lubricant; 0.1-8% taste regulating agents, 0-25% diluent; 0-15% disintegrant; 0-10% humectant; 0.2-6% coloring agent and/or flavoring agent, 10-60% effervescent acid and 15-50% effervescent base with respect to the total amount of unit dose with respect to the total amount of unit dose.

24. (canceled)

25. A pharmaceutical composition comprising ceftibuten, characterized in that at least one non-cellulose based binder having an average particle size in the range of 60-150 μm is used and the ratio of ceftibuten to said binder is in the range of 7:1 to 4:1.

26. (canceled)

27. The pharmaceutical composition according to claim 25, wherein ceftibuten is in the form of dihydrate and/or trihydrate.

28. (canceled)

29. The pharmaceutical composition according to claim 25, wherein povidone is used as the binder.

30. The pharmaceutical composition according to claim 25, wherein the amount of the binder is in the range of 1-5% of the total unit dose weight.

31. (canceled)

32. The pharmaceutical composition according to claim 25, wherein said composition further comprises lubricants, disintegrants, diluents, taste regulating agents, effervescent acid and effervescent base as pharmaceutically acceptable excipients.

33-37. (canceled)

38. The pharmaceutical composition according to claim 32, wherein saccharose, aspartame, sodium chloride or a combination thereof is used as the taste regulating agent.

39. The formulation according to claim 32, wherein 1-8% taste regulating agent is used with respect to the unit dose weight.

40. The formulation according to claim 32, wherein the ratio of ceftibuten to the taste regulating agent is in the range of 1:1 to 4:1.

41. The pharmaceutical composition according to claim 40, wherein the ratio of ceftibuten to the taste regulator is 3:1.

42-44. (canceled)

45. The pharmaceutical composition according to claim 25, wherein said composition comprises 5-50% ceftibuten; 1-5% binder; 0.1-8% lubricant; 0-15% disintegrant and/or disintegrants; 0-55% diluent; 1-8% taste regulating agent; 0.5-5% flavoring agents and 10-45% effervescent acid and 20-75% effervescent base with respect to the total amount of unit dose.

46. (canceled)

47. A formulation in suspension form comprising cefuroxime axetil characterized in that at least two pH agents are used and the ratio of the first pH agent having 46-100% (w/w) water solubility at 25° C. and the second pH agent having 1-45% (w/w) water solubility at 25° C. is in the range of 15:1 and 1:1 by weight.

48. (canceled)

49. The formulation according to claim 47, wherein the first pH agent is selected from a group comprising acetic acid, citric acid, potassium citrate, carbonic acid, hydrochloric acid, monosodium glutamate, lactic acid, malic acid, propionic acid, sulfuric acid, tartaric acid sodium hydroxide or a combination thereof.

50. The formulation according to claim 49, wherein citric acid is used as the first pH agent.

51. The formulation according to claim 47, wherein the second pH agent is selected from a group comprising sodium acetate, trisodium citrate, phosphoric acid, glutamic acid, dicalcium phosphate, trisodium phosphate, calcium sulfate or a combination thereof.

52. The formulation according to claim 51, wherein trisodium citrate is used as the second pH agent.

53. (canceled)

54. The formulation according to claim 52, wherein the total amount of the pH agents is in the range of 3-9% by weight.

55. (canceled)

56. The formulation according to claim 47, wherein said formulation further comprises one or several pharmaceutically acceptable excipients including binders, lubricants, glidants, viscosity agents, disintegrants, diluents, preservatives, flavoring agents, sweeteners, coloring agents, surfactants, antifoam agents and stabilizing agents.

57. (canceled)

58. The formulation according to claim 56, wherein the amount of the viscosity agent that said formulation contains is in the range of 0.1-4% by weight.

59-66. (canceled)

67. The formulation according to claim 47, wherein said formulation comprises;
Cefuroxime axetil in the range of 1-30% by weight
pH agent in the range of 1-20% by weight
Viscosity agent in the range of 0.1-4% by weight
Lubricant in the range of 0-5% by weight
Sweetener in the range of 20-90% by weight
Flavoring agent in the range of %0.5-5 by weight
Glidant in the range of %0.1-3 by weight
Preservative in the range of %0.05-4 by weight

68. (canceled)

69. A water soluble pharmaceutical formulation comprising a third generation cephalosporin and clavulanic acid and/or derivatives thereof in which a third generation cephalosporin antibiotic is used in 2%-15% of the unit dose; two different pH agents wherein one pH agent has pKa in the range of 1-5 and the other has pKa in the range of 5.1-15 are used to form a buffer system and the ratio of the first pH agent having pKa value of 1-5 to the second pH agent having the pKa value of 5.1-15 is in the range of 3:1 to 1:1.

70-71. (canceled)

72. The pharmaceutical formulation according to claim 69, wherein ceftibuten or cefdinir is used as the third generation cephalosporin.

73-75. (canceled)

76. The pharmaceutical formulation according to claim 72, wherein ceftibuten is used in dihydrate and/or trihydrate form.

77. The pharmaceutical formulation according to claim 72, wherein cefdinir is used in free form.

78-80. (canceled)

81. The pharmaceutical formulation according to claim 69, wherein the first pH agent having pKa value in the range of 1-5 is selected from citric acid and malic acid.

82. The pharmaceutical formulation according to claim 81, wherein citric acid is used as the first pH agent.

83. The pharmaceutical formulation according to claim 69, wherein the second pH agent having pKa value in the range of 5.1-15 is selected from tribasic calcium phosphate, monosodium glutamate, potassium citrate, trisodium citrate, sodium hydroxide, dibasic sodium phosphate, monobasic sodium phosphate or combinations thereof.

84. The pharmaceutical formulation according to claim 83, wherein trisodium citrate is used as the second pH agent.

85. (canceled)

86. The pharmaceutical formulation according to claim 69, wherein sweeteners, preservative agents, viscosity agents, glidants, lubricants, disintegrants, diluents and flavoring agents can be used in said formulation apart from the third generation cephalosporin, potassium clavulanate and pH agents.

87-92. (canceled)

93. The pharmaceutical formulation according to claim 86, wherein the ratio of the third generation cephalosporin to xanthan gum is in the range of 7:1 to 15:1.

94-105. (canceled)

106. The pharmaceutical formulation according to claim 69, wherein said formulation comprises 2-15% a third generation cephalosporin, 5-30% potassium clavulanate, 0.1-4% glidant, 0.2-3% lubricant, 0-20% disintegrant and/or disintegrants, 0-15% diluent, 30-90% sweetener, 0.5-4% pH agent, 0.1-2% preservative agent, 0.2-4% viscosity agent and 0.1-5% flavoring agent with respect to the total weight of the unit dose.

107. (canceled)
